(19) United States
(12) Patent Application Publication
(10) Pub. No.: US 2010/0241188 A1
(43) Pub. Date: Sep. 23, 2010

(54) Percutaneous Electrical Treatment Of Tissue

(75) Inventors: Joseph P. Errico, Green Brook, NJ (US); James R. Pastena, Succasunna, NJ (US); Steven Mendez, Chester, NJ (US); Hecheng Hu, Cedar Grove, NJ (US); Arthur Ross, Mendham, NJ (US); Bruce Simon, Mountain Lakes, NJ (US); Richard P. Dickerson, Rockaway, NJ (US)

Correspondence Address: ELECTROCORE INC., 51 GILBRALTAR DRIVE, SUITE 2F, POWER MILL PLAZA, MORRIS PLAINS, NJ, 07950-1254, US

(73) Assignee: ElectroCore, Inc., Morris Plains, NJ (US)

(21) Appl. No.: 12/422,483

(22) Filed: Apr. 13, 2009

Related U.S. Application Data
(63) Continuation-in-part of application No. 12/408,131, filed on Mar. 20, 2009.

(51) Int. Cl. A61N 1/36

(52) U.S. Cl. 607/42; 607/72

(57) ABSTRACT

Devices, systems and methods for applying electrical impulse(s) to one or more selected nerves in or around the carotid sheath are described. An electrode assembly is introduced through a percutaneous penetration in a patient to a target location adjacent to, or in close proximity with, the carotid sheath. Once in position, one or more electrical impulses are applied through the electrode assembly to one or more selected nerves to stimulate, block or otherwise modulate the nerve(s) and acutely treat the patient's condition.

### CROSS REFERENCE TO RELATED APPLICATIONS

This application is a continuation-in-part application of co-pending U.S. patent application Ser. No. 12/408,131, titled Electrical Treatment of Bronchial Constriction, filed Mar. 20, 2009, the entire disclosure of which is hereby incorporated by reference. This application is also related to commonly assigned co-pending U.S. patent Ser. Nos. 11/555,142, 11/555,170, 11/592,095, 11/591,340, 11/591,768, 11/754,522, 11/735,709 and 12/246,605, the complete disclosures of which are incorporated herein by reference for all purposes.

### BACKGROUND OF THE INVENTION

The present invention relates to the field of delivery of electrical impulses (and/or fields) to bodily tissues for therapeutic purposes, and more specifically to percutaneous devices and methods for treating conditions mediated by selected nerves.

There are a number of treatments for various infirmities that require the destruction of otherwise healthy tissue in order to affect a beneficial effect. Malfunctioning tissue is identified, and then lesioned or otherwise compromised in order to affect a beneficial outcome, rather than attempting to repair the tissue to its normal functionality. While there are a variety of different techniques and mechanisms that have been designed to focus lesioning directly onto the target nerve tissue, collateral damage is inevitable.

Still other treatments for malfunctioning tissue can be medicinal in nature, in many cases leaving patients to become dependent upon artificially synthesized chemicals. Examples of this are anti-asthma drugs such as albuterol, proton pump inhibitors such as omeprazole (Prilosec), spastic bladder relievers such as Ditropan, and cholesterol reducing drugs like Lipitor and Zocor. In many cases, these medicinal approaches have side effects that are either unknown or quite significant, for example, at least one popular diet pill of the late 1990's was subsequently found to cause heart attacks and strokes.

Unfortunately, the beneficial outcomes of surgery and medicines are, therefore, often realized at the cost of function of other tissues, or risks of side effects.

The use of electrical stimulation for treatment of medical conditions has been well known in the art for nearly two thousand years. It has been recognized that electrical stimulation of the brain and/or the peripheral nervous system and/or direct stimulation of the malfunctioning tissue, which stimulation is generally a wholly reversible and non-destructive treatment, holds significant promise for the treatment of many ailments.

Electrical stimulation of the brain with implanted electrodes has been approved for use in the treatment of various conditions, including pain and movement disorders including essential tremor and Parkinson's disease. The principle behind these approaches involves disruption and modulation of hyperactive neuronal circuit transmission at specific sites in the brain. As compared with the very dangerous lesioning procedures in which the portions of the brain that are behaving pathologically are physically destroyed, electrical stimulation is achieved by implanting electrodes at these sites to, first sense aberrant electrical signals and then to send electrical pulses to locally disrupt the pathological neuronal transmission, driving it back into the normal range of activity. These electrical stimulation procedures, while invasive, are generally conducted with the patient conscious and a participant in the surgery.

Brain stimulation, and deep brain stimulation in particular, is not without some drawbacks. The procedure requires penetrating the skull, and inserting an electrode into the brain matter using a catheter-shaped lead, or the like. While monitoring the patient's condition (such as tremor activity, etc.), the position of the electrode is adjusted to achieve significant therapeutic potential. Next, adjustments are made to the electrical stimulus signals, such as frequency, periodicity, voltage, current, etc., again to achieve therapeutic results. The electrode is then permanently implanted and wires are directed from the electrode to the site of a surgically implanted pacemaker. The pacemaker provides the electrical stimulus signals to the electrode to maintain the therapeutic effect. While the therapeutic results of deep brain stimulation are promising, there are significant complications that arise from the implantation procedure, including stroke induced by damage to surrounding tissues and the neurovasculature.

One of the most successful modern applications of this basic understanding of the relationship between muscle and nerves is the cardiac pacemaker. Although its roots extend back into the 1800's, it was not until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed.

Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

Another application of electrical stimulation of nerves has been the treatment of radiating pain in the lower extremities by means of stimulation of the sacral nerve roots at the bottom of the spinal cord (see U.S. Pat. No. 6,871,099 to Whitehurst, et al., the disclosure of which is incorporated herein by reference).

The smooth muscles that line the bronchial passages are controlled by a confluence of vagus and sympathetic nerve fiber plexuses. Spasms of the bronchi during asthma or COPD attacks and anaphylactic shock can often be directly related to pathological signaling within these plexuses. Anaphylactic shock, COPD and asthma are major health concerns.

Asthma, and other airway occluding disorders resulting from inflammatory responses and inflammation-mediated bronchoconstriction, affects an estimated eight to thirteen million adults and children in the United States. A significant subclass of asthmatics suffers from severe asthma. An estimated 5,000 persons die every year in the United States as a result of asthma attacks. Up to twenty percent of the populations of some countries are affected by asthma, estimated at more than a hundred million people worldwide. Asthma's associated morbidity and mortality are rising in most countries despite increasing use of anti-asthma drugs.

Asthma is characterized as a chronic inflammatory condition of the airways. Typical symptoms are coughing, wheezing, tightness of the chest and shortness of breath. Asthma is a result of increased sensitivity to foreign bodies such as pollen, dust mites and cigarette smoke. The body, in effect, overreacts to the presence of these foreign bodies in the airways. As part of the asthmatic reaction, an increase in mucous production is often triggered, exacerbating airway restriction. Smooth muscle surrounding the airways goes into spasm, resulting in constriction of airways. The airways also become inflamed. Over time, this inflammation can lead to scarring of the airways and a further reduction in airflow. This inflammation leads to the airways becoming more irritable, which may cause an increase in coughing and increased susceptibility to asthma episodes.

Two medicinal strategies exist for treating this problem for patients with asthma. The condition is typically managed by means of inhaled medications that are taken after the onset of symptoms, or by injected and/or oral medication that are taken chronically. The medications typically fall into two categories; those that treat the inflammation, and those that treat the smooth muscle constriction. The first is to provide anti-inflammatory medications, like steroids, to treat the airway tissue, reducing its tendency to over-release of the molecules that mediate the inflammatory process. The second strategy is to provide a smooth muscle relaxant (e.g. an anti-cholinergic) to reduce the ability of the muscles to constrict.

It has been highly preferred that patients rely on avoidance of triggers and anti-inflammatory medications, rather than on the bronchodilators as their first line of treatment. For some patients, however, these medications, and even the bronchodilators are insufficient to stop the constriction of their bronchial passages, and more than five thousand people suffocate and die every year as a result of asthma attacks.

Anaphylaxis likely ranks among the other airway occluding disorders of this type as the most deadly, claiming many deaths in the United States every year. Anaphylaxis (the most severe form of which is anaphylactic shock) is a severe and rapid systemic allergic reaction to an allergen. Minute amounts of allergens may cause a life-threatening anaphylactic reaction. Anaphylaxis may occur after ingestion, inhalation, skin contact or injection of an allergen. Anaphylactic shock usually results in death in minutes if untreated. Anaphylactic shock is a life-threatening medical emergency because of rapid constriction of the airway. Brain damage sets in quickly without oxygen.

The triggers for these fatal reactions range from foods (nuts and shellfish), to insect stings (bees), to medication (radio contrasts and antibiotics). It is estimated 1.3 to 13 million people in the United States are allergic to venom associated with insect bites; 27 million are allergic to antibiotics; and 5-8 million suffer food allergies. All of these individuals are at risk of anaphylactic shock from exposure to any of the foregoing allergens. In addition, anaphylactic shock can be brought on by exercise. Yet all are mediated by a series of hypersensitivity responses that result in uncontrollable airway occlusion driven by smooth muscle constriction, and dramatic hypotension that leads to shock. Cardiovascular failure, multiple organ ischemia, and asphyxiation are the most dangerous consequences of anaphylaxis.

Anaphylactic shock requires advanced medical care immediately. Current emergency measures include rescue breathing; administration of epinephrine; and/or intubation if possible. Rescue breathing may be hindered by the closing airway but can help if the victim stops breathing on his own. Clinical treatment typically consists of antihistamines (which inhibit the effects of histamine at histamine receptors) which are usually not sufficient in anaphylaxis, and high doses of intravenous corticosteroids. Hypotension is treated with intravenous fluids and sometimes vasoconstrictor drugs. For bronchospasm, bronchodilator drugs such as salbutamol are employed.

Given the common mediators of both asthmatic and anaphylactic bronchoconstriction, it is not surprising that asthma sufferers are at a particular risk for anaphylaxis. Still, estimates place the numbers of people who are susceptible to such responses at more than 40 million in the United States alone.

Tragically, many of these patients are fully aware of the severity of their condition, and die while struggling in vain to manage the attack medically. Many of these incidents occur in hospitals or in ambulances, in the presence of highly trained medical personnel who are powerless to break the cycle of inflammation and bronchoconstriction (and life-threatening hypotension in the case of anaphylaxis) affecting their patient.

Unfortunately, prompt medical attention for anaphylactic shock and asthma are not always available. For example, epinephrine is not always available for immediate injection. Even in cases where medication and attention is available, life saving measures are often frustrated because of the nature of the symptoms. Constriction of the airways frustrates resuscitation efforts, and intubation may be impossible because of swelling of tissues.

Typically, the severity and rapid onset of anaphylactic reactions does not render the pathology amenable to chronic treatment, but requires more immediately acting medications. Among the most popular medications for treating anaphylaxis is epinephrine, commonly marketed in so-called “Epi-pen” formulations and administering devices, which potential sufferers carry with them at all times. In addition to serving as an extreme bronchodilator, epinephrine raises the patient's heart rate dramatically in order to offset the hypotension that accompanies many reactions. This cardiovascular stress can result in tachycardia, heart attacks and strokes.

Chronic obstructive pulmonary disease (COPD) is a major cause of disability, and is the fourth leading cause of death in the United States. More than 12 million people are currently diagnosed with COPD. An additional 12 million likely have the disease and don't even know it. COPD is a progressive disease that makes it hard for the patient to breathe. COPD can cause coughing that produces large amounts of mucus, wheezing, shortness of breath, chest tightness and other symptoms. Cigarette smoking is the leading cause of COPD, although long-term exposure to other lung irritants, such as air pollution, chemical fumes or dust may also contribute to COPD. In COPD, less air flows in and out of the bronchial airways for a variety of reasons, including loss of elasticity in the airways and/or air sacs, inflammation and/or destruction of the walls between many of the air sacs and overproduction of mucus within the airways.

The term COPD includes two primary conditions: emphysema and chronic obstructive bronchitis. In emphysema, the walls between many of the air sacs are damaged, causing them to lose their shape and become floppy. This damage also can destroy the walls of the air sacs, leading to fewer and larger air sacs instead of many tiny ones. In chronic obstructive bronchitis, the patient suffers from permanently irritated and inflamed bronchial tissue that is slowly and progressively dying. This causes the lining to thicken and form thick mucus, making it hard to breathe. Many of these patients also experience periodic episodes of acute airway reactivity (i.e., acute exacerbations), wherein the smooth muscle surrounding the airways goes into spasm, resulting in further constriction and inflammation of the airways. Acute exacerbations occur, on average, between two and three times a year in patients with moderate to severe COPD and are the most common cause of hospitalization in these patients (mortality rates are 11%). Frequent acute exacerbations of COPD cause lung function to deteriorate quickly, and patients never recover to the condition they were in before the last exacerbation. Similar to asthma, current medical management of these acute exacerbations is often insufficient.

Unlike cardiac arrhythmias, which can be treated chronically with pacemaker technology, or in emergent situations with equipment like defibrillators (implantable and external), there is virtually no commercially available medical equipment that can chronically reduce the baseline sensitivity of the muscle tissue in the airways to reduce the predisposition to asthma attacks, reduce the symptoms of COPD or to break the cycle of bronchial constriction associated with an acute asthma attack or anaphylaxis.

Accordingly, there is a need in the art for new products and methods for acutely treating the immediate symptoms of certain conditions, such as bronchial constriction resulting from pathologies such as anaphylactic shock, asthma and COPD.

### SUMMARY OF THE INVENTION

The present invention provides systems, apparatus and methods for selectively applying electrical energy to body tissue. The invention is particularly useful for immediately treating an acute condition or symptom of a patient.

In one aspect of the invention, an electrode assembly is introduced through a percutaneous penetration in a patient to a target location adjacent to, or in close proximity with, the carotid sheath. Once in position, an electrical impulse is applied through the electrode assembly to one or more selected nerves to stimulate, block or otherwise modulate the nerve(s) and acutely treat the patient's condition or a symptom of that condition. As used herein, the term acutely means that the electrical impulse immediately begins to interact with one or more nerves to produce a response in the patient. In certain embodiments, the electrical impulse will produce a response in the nerve(s) to improve the patient's condition or symptom in less than 3 hours, preferably less than 1 hour and more preferably less than 15 minutes.

In a preferred embodiment, an access device, such as a needle, is introduced through the patient's skin surface in the neck and advanced to the target location proximal to the carotid sheath. The target location may be directly adjacent to or in contact with the carotid sheath, or it may be in close proximity with (e.g., within 1-5 mm) of the carotid sheath. The exact location of the target location will depend on the configuration of the electrode assembly, the strength or amplitude of the electrical impulse and the actual condition of the patient. Once the access device is in position, the electrode assembly is advanced to the target location and secured in position, preferably parallel to the carotid sheath. In certain embodiments, a cannula or similar device is first advanced to the target region and the electrode assembly is then directed through the cannula. In other embodiments, the electrode assembly may include an insulating sheath that allows the electrodes to be directly advanced to the target region (i.e., without the use of a cannula).

In one embodiment, the electrode assembly comprises an active and a return electrode located at the distal end of a flexible electrical lead. In this embodiment, an electrical impulse is applied across the active and return electrodes such that the electric current is generally confined within a local space around the electrode assembly (i.e., a bipolar electrode assembly). In other embodiments, the return electrode is a return pad located on a surface of the patient's skin, such as the back or hip, and the electrode at the distal end portion of the flexible lead acts as the tissue treatment or active electrode (i.e., a monopolar electrode assembly). It will be understood by those in the art that other configurations are possible, such as multiple active or tissue treatment electrodes and/or multiple return electrodes.

In a preferred embodiment, the source of electrical energy is an electrical signal generator that preferably operates to generate an electrical signal having a frequency between about 1 Hz to 3000 Hz, a pulse duration of between about 10-1000 us, and an amplitude of between about 1-20 volts. The electrical signal may be one or more of: a full or partial sinusoid, a square wave, a rectangular wave, and triangle wave. By way of example, the at least one electrical signal may be of a frequency between about 15 Hz to 35 Hz. By way of example, the at least one electrical signal may have a pulsed on-time of between about 50 to 1000 microseconds, such as between about 100 to 300 microseconds, or about 200 microseconds. By way of example, the at least one electrical signal may have an amplitude of about 5-15 volts, such as about 12 volts.

In another aspect of the invention, a device for acutely treating a patient's condition includes a source of electrical energy and an electrode assembly configured for percutaneous delivery of an electrical impulse(s) to a target region in or around the carotid sheath of the patient. The electrical impulse is sufficient to modulate a selected nerve at or around the target region to acutely treat a condition or symptom of the patient. The device preferably includes an introducer for creating percutaneous access to the target region. The introducer may include an access device, such as a needle, for creating percutaneous access through a skin surface of the patient's neck and a cannula having an inner lumen for passage of the electrode assembly therethrough. The electrode assembly preferably includes an active electrode, a return electrode and flexible electrical leads coupling the active and return electrodes to the source of electrical energy.

In accordance with one aspect of the invention, a method is provided to percutaneously apply an electrical impulse to modulate, stimulate, inhibit or block electrical signals in nerves within or around the carotid sheath, such as parasympathetic and/or sympathetic nerves, to acutely treat a condition or symptom of a patient. In one embodiment, the electrical impulse is sufficient to acutely reduce the magnitude of constriction of bronchial smooth muscle of a patient. One or more aspects of the present invention are particularly useful for the acute relief of symptoms associated with bronchial constriction, i.e., asthma attacks, COPD exacerbations and/or anaphylactic reactions. The teachings of various aspects of the present invention provide an emergency response to such acute symptoms, by producing immediate airway dilation and/or heart function increase to enable subsequent adjunctive measures (such as the administration of epinephrine) to be effectively employed.

In another embodiment, methods and apparatus for treating the temporary arrest of intestinal peristalsis, such as post-operative ileus, are provided. In this embodiment, an electrode assembly is introduced through the patient's neck and advanced to the target region in or around the carotid sheath as described above. Electrical signals are applied to the electrode assembly to modulate, stimulate and/or block nerve signals thereof such that intestinal peristalsis function is at least partially improved.

In yet another embodiment of the present invention, the treatment of hypotension may be achieved utilizing an electrical signal that may be applied to selected nerves in the carotid sheath, such as the vagus nerve, to temporarily stimulate, block and/or modulate the signals in the selected nerves. Embodiments of the present invention also encompass treatment of pathologies causing hypotension, both chronic and acute hypotension, such as in patients with thyroid pathologies and those suffering from septic shock. The electrical impulses may be applied to at least one selected region of the carotid sheath to stimulate, block and/or modulate signals to the smooth muscle surrounding blood vessels, causing them to constrict and raise blood pressure.

Other embodiments of the present invention are useful as an acute testing device to determine if longer term or permanent implantable devices will have the desired effect on a patient. For example, the electrical signal may be adapted to reduce, stimulate, inhibit or block electrical signals in the vagus nerve to treat many known vagal nerve stimulation applications, such as hypotension associated with sepsis or anaphylaxis, hypertension, diabetes, hypovolemic shock, asthma, sepsis, epilepsy, depression, obesity, anxiety disorders, migraines, Alzheimer's disease and any other ailment affected by vagus nerve transmissions.

The novel systems, devices and methods of the present invention are more completely described in the following detailed description of the invention, with reference to the drawings provided herewith, and in claims appended hereto. Other aspects, features, advantages, etc. will become apparent to one skilled in the art when the description of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited by or to the precise data, methodologies, arrangements and instrumentalities shown, but rather only by the claims.

FIG. 1 is a schematic view of a nerve modulating device according to one or more aspects of the present invention;

FIG. 2 illustrates an exemplary electrical voltage/current profile for a blocking and/or modulating impulse applied to a portion or portions of a nerve in accordance with an embodiment of the present invention;

FIG. 3 illustrates the major vessels of the neck, including the carotid sheath;

FIG. 4 illustrates an electrode assembly according to one embodiment of the invention;

FIG. 5 illustrates an introducer according to one embodiment of the present invention;

FIG. 6 illustrates the introducer of FIG. 5 as it is advanced through a percutaneous penetration in a patient to the target region near the carotid sheath;

FIG. 7 illustrates the electrode assembly of FIG. 4 as it is advanced through the introducer to the target region in the patient;

FIG. 8 illustrates an exemplary connector for coupling the electrode assembly of FIG. 4 to a source of electrical energy (not shown);

FIG. 9 illustrates removal of the introducer and the electrode assembly and connector after said removal, respectively; and

FIGS. 10-13 graphically illustrate exemplary experimental data obtained on human patients in accordance with one embodiment of the present invention.

### DETAILED DESCRIPTION OF THE PREFERRED EMBODIMENTS

In one or more embodiments of the present invention, electrical energy is applied through a percutaneous penetration in a patient to a target region around the carotid sheath to acutely treat a patient's ailment. The invention is particularly useful for applying electrical impulses that interact with the signals of one or more nerves, or muscles, to achieve a therapeutic result, such as relaxation of the smooth muscle of the bronchia, increase in blood pressure associated with orthostatic hypotension, reduction in blood pressure, treatment of epilepsy, treating ileus conditions, depression, anaphylaxis, obesity, and/or any other ailment affected by nerve transmissions, such as the vagus nerve or the spinal cord. In particular, embodiments of the present invention can be used to practice the treatments described in the following commonly assigned patent applications: US Patent Publication Numbers: 2009/0183237, 2008/0009913, 2007/0191902, 2007/0191905, 2007/0106339, 2007/0106338 and 2007/0106337, the full disclosures of which were previously incorporated herein by reference.

For convenience, the remaining disclosure will be directed specifically to the treatment of acute bronchoconstriction, but it will be appreciated that the systems and methods of the present invention can be applied equally well to other tissues and nerves of the body, including but not limited to other parasympathetic nerves, sympathetic nerves, spinal or cranial nerves, e.g., optic nerve, facial nerves, vestibulocochlear nerves and the like. In addition, embodiments of the present invention can be applied to treat other symptoms of ailments or the ailments themselves, such as asthma, COPD, sepsis, dialytic hypotension, epilepsy, depression or obesity and other procedures including open procedures, intravascular procedures, interventional cardiology procedures, urology, laparoscopy, general surgery, arthroscopy, thoracoscopy or other cardiac procedures, cosmetic surgery, orthopedics, gynecology, otorhinolaryngology, spinal and neurologic procedures, oncology procedures and the like.

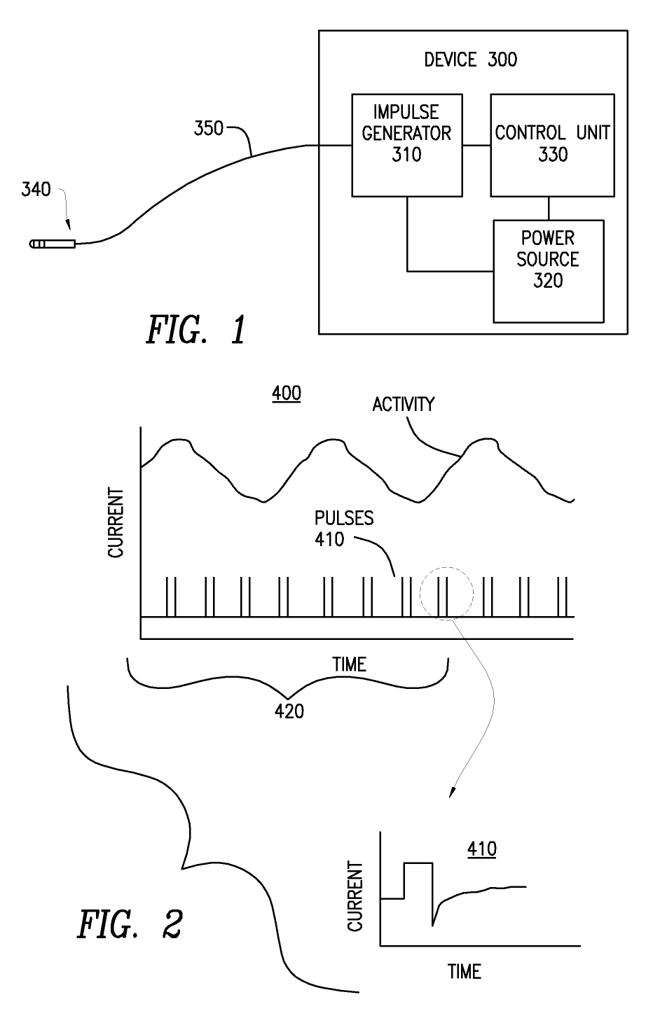

FIG. 1 is a schematic diagram of a nerve modulating device 300 for delivering electrical impulses to nerves. As shown, device 300 may include an electrical impulse generator 310; a power source 320 coupled to the electrical impulse generator 310; a control unit 330 in communication with the electrical impulse generator 310 and coupled to the power source 320; and an electrode assembly 340 coupled to the electrical impulse generator 310 for attachment via lead 350 to one or more selected regions of a nerve (not shown). The control unit 330 may control the electrical impulse generator 310 for generation of a signal suitable for amelioration of a patient's condition when the signal is applied via the electrode assembly 340 to the nerve. It is noted that nerve modulating device 300 may be referred to by its function as a pulse generator. U.S. Patent Application Publications 2005/0075701 and 2005/0075702, both to Shafer, both of which are incorporated herein by reference, relating to stimulation of neurons of the sympathetic nervous system to attenuate an immune response, contain descriptions of pulse generators that may be applicable to various embodiments of the present invention.

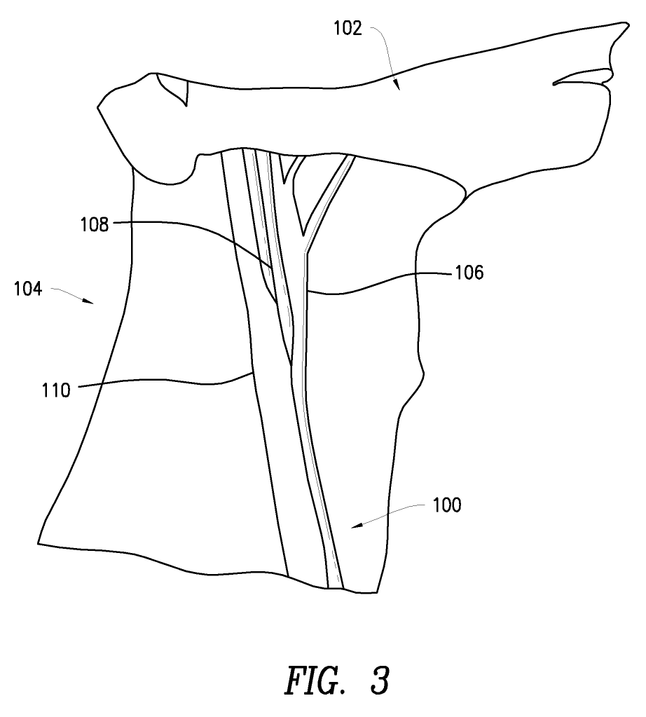

FIG. 2 illustrates an exemplary electrical voltage/current profile for a stimulating, blocking and/or modulating impulse applied to a portion or portions of selected nerves in accordance with an embodiment of the present invention. As shown, a suitable electrical voltage/current profile 400 for the blocking and/or modulating impulse 410 to the portion or portions of a nerve may be achieved using pulse generator 310. In a preferred embodiment, the pulse generator 310 may be implemented using a power source 320 and a control unit 330 having, for instance, a processor, a clock, a memory, etc., to produce a pulse train 420 to the electrode(s) 340 that deliver the stimulating, blocking and/or modulating impulse 410 to the nerve via lead 350. Nerve modulating device 300 may be powered and/or recharged from outside the body or may have its own power source 320. By way of example, device 300 may be purchased commercially. Nerve modulating device 300 is preferably programmed with a physician programmer, such as a Model 7432 also available from Medtronic, Inc.

The parameters of the modulation signal 400 are preferably programmable, such as the frequency, amplitude, duty cycle, pulse width, pulse shape, etc. In the case of an implanted pulse generator, programming may take place before or after implantation. For example, an implanted pulse generator may have an external device for communication of settings to the generator. An external communication device may modify the pulse generator programming to improve treatment.

In addition, or as an alternative to the devices to implement the modulation unit for producing the electrical voltage/current profile of the stimulating, blocking and/or modulating impulse to the electrodes, the device disclosed in U.S. Patent Publication No.: 2005/0216062 (the entire disclosure of which is incorporated herein by reference), may be employed. U.S. Patent Publication No.: 2005/0216062 discloses a multi-functional electrical stimulation (ES) system adapted to yield output signals for effecting, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or a saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated.

The electrical leads 350 and electrodes 340 are preferably selected to achieve respective impedances permitting a peak pulse voltage in the range from about 0.2 volts to about 20 volts.

The stimulating, blocking and/or modulating impulse signal 410 preferably has a frequency, an amplitude, a duty cycle, a pulse width, a pulse shape, etc. selected to influence the therapeutic result, namely stimulating, blocking and/or modulating some or all of the transmission of the selected nerve. For example the frequency may be about 1 Hz or greater, such as between about 15 Hz to 50 Hz, more preferably around 25 Hz. The modulation signal may have a pulse width selected to influence the therapeutic result, such as about 20 μS or greater, such as about 20 μS to about 1000 μS. The modulation signal may have a peak voltage amplitude selected to influence the therapeutic result, such as about 0.2 volts or greater, such as about 0.2 volts to about 20 volts.

In a preferred embodiment of the invention, a method of treating bronchial constriction comprises applying one or more electrical impulse(s) of a frequency of about 15 Hz to 50 Hz to a selected region of the carotid sheath to reduce a magnitude of constriction of bronchial smooth muscle. As discussed in more detail below, applicant has made the unexpected discovered that applying an electrical impulse to a selected region of the carotid sheath within this particular frequency range results in almost immediate and significant improvement in bronchodilation, as discussed in further detail below. Applicant has further discovered that applying electrical impulses outside of the selected frequency range (15 Hz to 50 Hz) does not result in immediate and significant improvement in bronchodilation. Preferably, the frequency is about 25 Hz. In this embodiment, the electrical impulse(s) are of an amplitude of between about 0.75 to 12 volts (depending on the size and shape of the electrodes and the distance between the electrodes and the selected nerve(s)) and have a pulsed on-time of between about 50 to 500 microseconds, preferably about 200-400 microseconds.

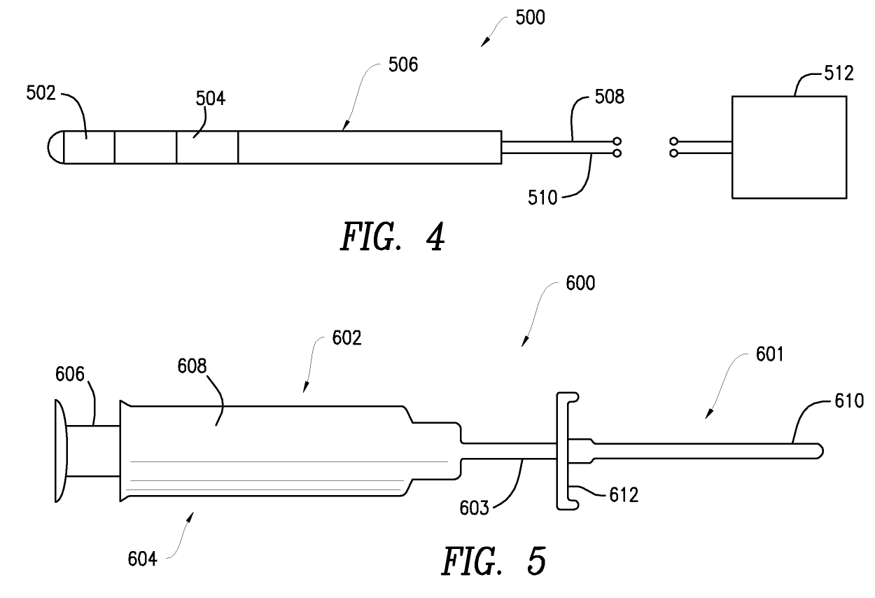

FIG. 3 illustrates some of the major structures of the neck. As shown, the common carotid artery 100 extends from the base of the skull 102 through the neck 104 to the first rib and sternum (not shown). Carotid artery 100 includes an external carotid artery 106 and an internal carotid artery 108 and is protected by fibrous connective tissue called the carotid sheath. The carotid sheath is located at the lateral boundary of the retopharyngeal space at the level of the oropharynx on each side of the neck 104 and deep to the sternocleidomastoid muscle. The three major structures within the carotid sheath are the common carotid artery 100, the internal jugular vein 110 and the vagus nerve (not shown). The carotid artery lies medial to the internal jugular vein and the vagus nerve is situated posteriorly between the two vessels.

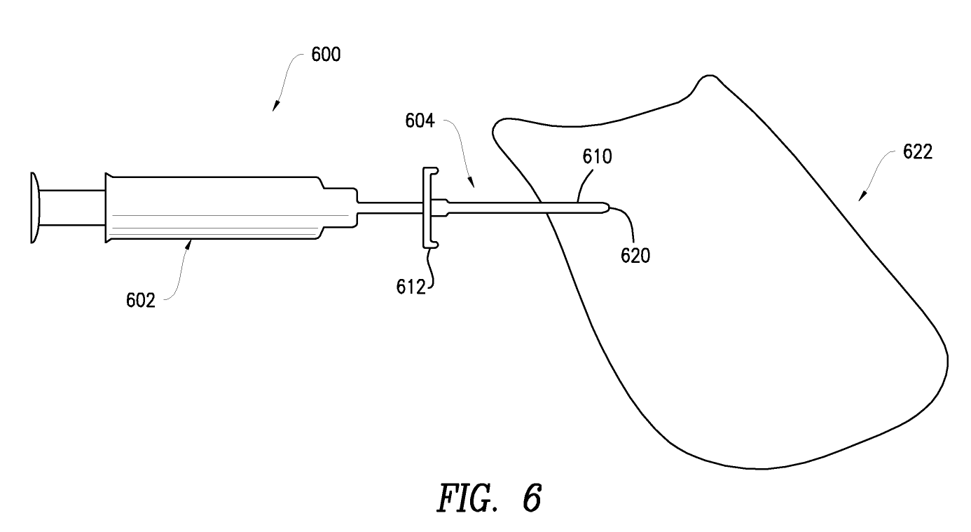

FIG. 4 illustrates an exemplary electrode assembly 500 according to one embodiment of the present invention. As shown, electrode assembly 500 includes an active electrode 502 and a return electrode 504 coupled to the distal end of an insulating flexible shaft 506. The active and return electrodes 502, 504 have leads 508, 510, respectively, extending through shaft 508 for coupling the electrodes to a connector block 512 proximal to the shaft 508. Active and return electrodes 502, 504 are spaced a suitable distance to allow for the formation of an electromagnetic field around electrode assembly 500 for modulation of nerve (s) at the target region (not shown). In this embodiment, electrodes 502, 504 are spaced from each other by about 5-50 mm, preferably between about 10-20 mm.

Although there are a number of sizes and shapes that would suffice to implement electrodes 502, 504, by way of example, electrodes may be between about 1.0-1.5 mm long (such as 1.2 mm), may have an outside diameter of between about 2.6-2.85 mm (such as 2.7 mm), and may have an inside diameter of between about 2.5-2.75 mm (such as 2.7 mm). A suitable electrode may be formed from Pt-IR (90%/10%), although other materials or combinations or materials may be used, such as platinum, tungsten, gold, copper, palladium, silver or the like. Although the specific implementation of electrode assembly is not of criticality to the invention, by way of example, suitable electrode assemblies may be purchased commercially from Ad-Tech Medical in Racine, Wis.

Those skilled in the art will also recognize that a variety of different shapes and sizes of electrodes may be used. By way of example only, electrode shapes according to various aspects of the present invention can include ball shapes, twizzle shapes, spring shapes, twisted metal shapes, annular, solid tube shapes or the like. Alternatively, the electrode(s) may comprise a plurality of filaments, rigid or flexible brush electrode(s), coiled electrode(s) or the like. Alternatively, the electrode may be formed by the use of formed wire (e.g., by drawing round wire through a shaping die) to form electrodes with a variety of cross-sectional shapes, such as square, rectangular, L or V shaped, or the like.

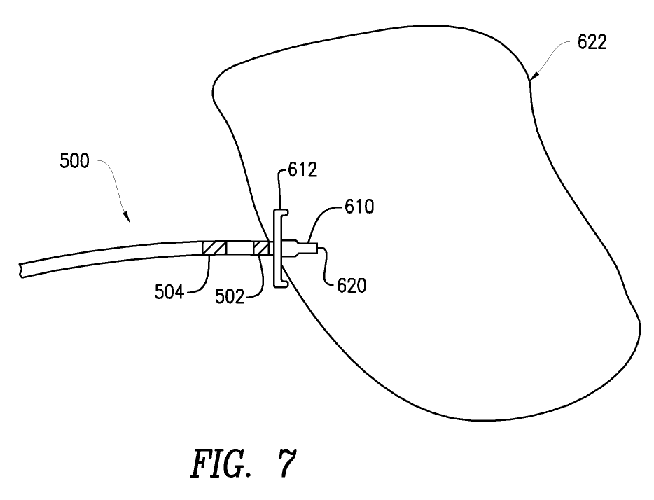

FIG. 5 illustrates an exemplary introducer 600 according to one embodiment of the present invention. As shown, introducer 600 includes a needle assembly 602 and a sheath or cannula 601. In this embodiment, needle assembly 602 is a syringe having a hypodermic needle 603 coupled to a piston pump 604 with a plunger 606 that fits within a cylindrical hollow tube 608. As is well known in the art, plunger 606 can be pulled and pushed along the inside of tube 608 to take in and expel liquids or gases through an orifice (not shown) at the open end of tube 608. Cannula 601 includes a base 612 and a hollow tube 610 sized to receive hypodermic needle 603 and electrode assembly 500 (as discussed below). Although the specific cannula used is not of criticality to the invention, suitable cannulas can be purchased commercially from Epimed.

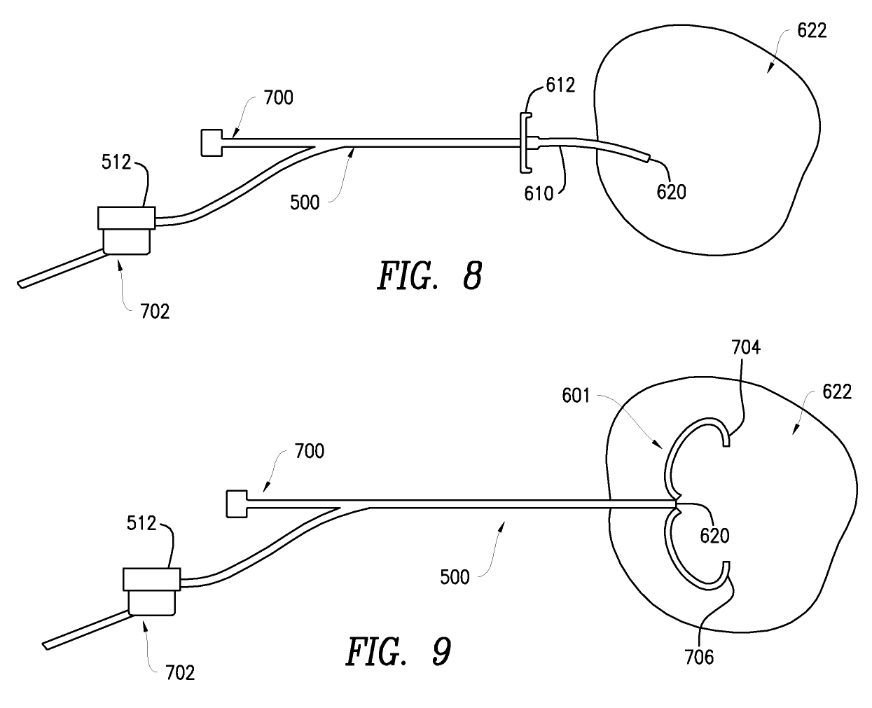

FIGS. 6-9 illustrate a method of applying an electrical impulse to the carotid sheath of a patient according to one or more aspects of the present invention. Typically, the carotid sheath or jugular vein will be located in any manner known in the art, e.g., by feel or ultrasound. Once the patient is prepared for the procedure, the target area of the skin on the neck is anesthetized (e.g., with lidocaine or a similar local anestheia). The target area may be any suitable location that will allow for access to the carotid sheath.

In one embodiment, a finder needle (not shown) may be used to first locate the target region around the carotid sheath. The finder needle is preferably a small access needle having a size in the range of 18-26 gauge, preferably around 22 gauge. Suitable finder needles for use in one or more embodiments of the present invention may be purchased commercially from Epimed. Typically, the finder needle is inserted through the skin surface and advanced to approach the carotid sheath. In certain embodiments, nerves extending through the carotid sheath, such as the vagus nerve, are targeted for modulation. An excitable tissue cell, such as a nerve fiber, is substantially less sensitive to a transverse electric field than a longitudinal electric field. Applying a longitudinal field increases the effect of this field on the excitable cell at the same frequencies, amplitudes, pulse durations and power levels. Thus, in these embodiments, the finder needle is preferably advanced to approach the carotid sheath in parallel. In other embodiments, the finder needle may be advanced to positions transverse to the carotid sheath.

The finder needle may be aspirated at this point to ensure that it has not penetrated the jugular vein or carotid artery. Alternatively, ultrasound may be used to verify the exact placement of the finder needle. Once the finder needle is in place, an additional incision may be made, e.g. with a scalpel, to provide access to introducer 600. In alternative embodiments, introducer 600 may be directly inserted into patient without the use of a finder needle as described above. As shown in FIG. 6, tube 610 of introducer 600 is driven through a percutaneous penetration 620 in the neck 622 of a patient and advanced along the same entry path as the finder needle until it reaches the desired depth of placement of the target region proximal to the carotid sheath. The physician may also aspirate needle 603 to ensure that it has not penetrated into a venous or arterial structure. Needle assembly 602 is then removed from cannula 601 by pressing against base 612 while needle assembly 602 is withdrawn.

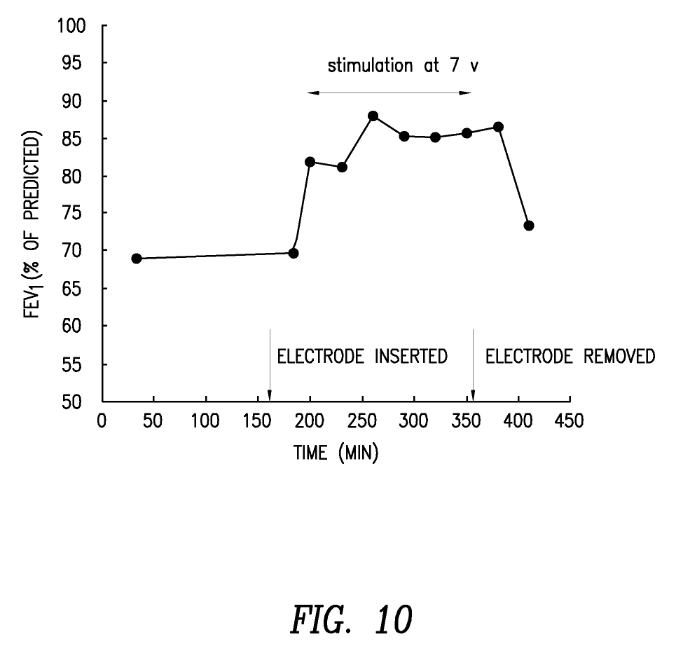

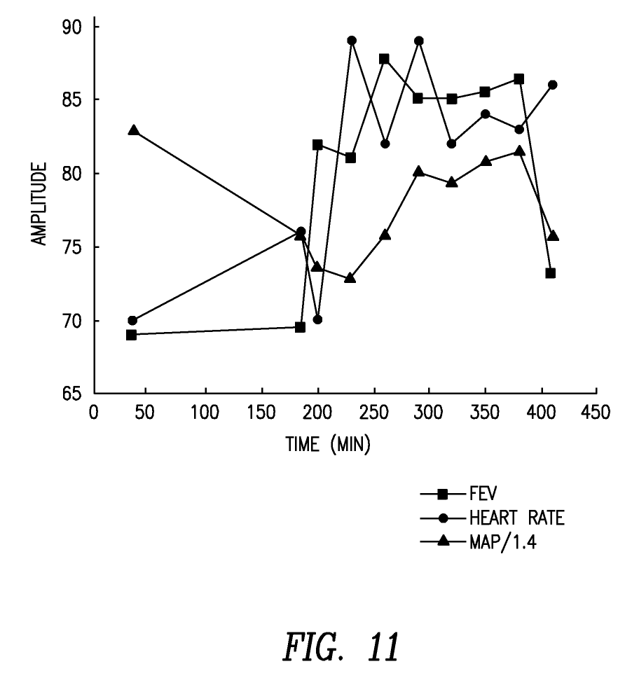

Referring now to FIG. 7, electrode assembly 500 may now be inserted into cannula 601 and advanced to the target region. As shown, the distal end portion of electrode assembly 500 is sized to fit and easily slide through the inner lumen of cannula 610 such that active and return electrodes 502, 504 can be located at the desired depth/position parallel to the carotid sheath. As shown in FIG. 8, a delivery stylet 700 may be used to provide rigidity to the flexible shaft of electrode assembly 500 to assist with the insertion process. A suitable delivery stylet may be purchased commercially from AD-Tech. Of course, it will be recognized by those skilled in the art that electrode assembly 500 may be advanced to the target region in a variety of manners other than stylet 700. Once in place, connector block 512 is attached to a cable 702 to electrically couple electrode assembly 500 to a source of electrical energy (not shown). At this point, the system may be tested to ensure proper functioning by activating the source of electrical energy and noting any muscle tremor at the target region.

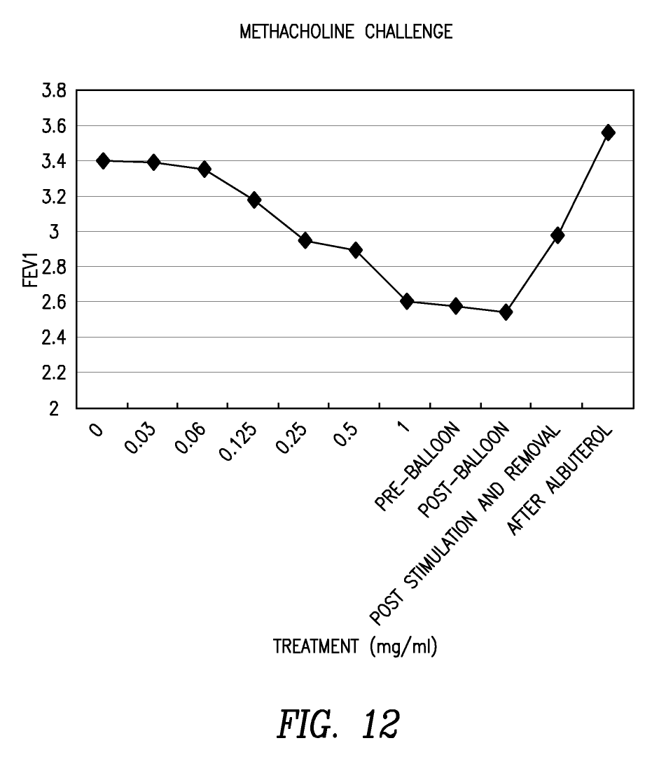

As shown in FIG. 9, cannula 601 may now be removed from the patient. In one embodiment, this is accomplished by bending tabs 704, 706 of base 612 downward and pulling them apart, thereby splitting cannula 601 into two pieces. Cannula 601 is then removed while electrode assembly 500 is held securely to prevent migration during cannula 601 removal. Similarly, delivery stylet 700 may be removed from patient leaving only the electrode assembly 500 in position at the target region. Electrode assembly 500 is then secured in place on the patient, e.g., with the use of tape or sutures (not shown), to ensure that is it does not migrate during the procedure. Alternatively, stylet 700 and/or cannula 601 may be left in place during the entire procedure.

In one specific embodiment, methods and devices of the present invention are particularly useful for providing substantially immediate relief of acute symptoms associated with bronchial constriction such as asthma attacks, COPD exacerbations and/or anaphylactic reactions. One of the advantages of the present invention is the ability to provide almost immediate dilation of the bronchial smooth muscle in patients suffering from acute bronchoconstriction, opening the patient's airways and allowing them to breathe and more quickly recover from an acute episode (i.e., a relatively rapid onset of symptoms that are typically not prolonged or chronic).

The magnitude of bronchial constriction in a patient is typically expressed in a measurement referred to as the Forced Expiratory Volume in 1 second (FEV₁). FEV₁ represents the amount of air a patient exhales (expressed in liters) in the first second of a pulmonary function test, which is typically performed with a spirometer. The spirometer compares the FEV₁ result to a standard for the patient, which is based on the predicted value for the patient's weight, height, sex, age and race. This comparison is then expressed as a percentage of the FEV₁ as predicted. Thus, if the volume of air exhaled by a patient in the first second is 60% of the predicted value based on the standard, the FEV₁ will be expressed in both the actual liters exhaled and as a percentage of predicted (i.e., 60% of predicted).

As will be discussed in more detail in the experiments below, applicants have disclosed a system and method for increasing a patient's FEV₁ in a relatively short period of time. Preferably, the electrical impulse applied to the patient is sufficient to increase the FEV₁ of the patient by a clinically significant amount in a period of time less than about 6 hours, preferably less than 3 hours and more preferably less than 90 minutes. In an exemplary embodiment, the clinically significant increase in FEV₁ occurs in less than 15 minutes. A clinically significant amount is defined herein as at least a 12% increase in the patient's FEV₁ versus the FEV₁ prior to application of the electrical impulse.

Prior to discussing experimental results, a general approach to treating bronchial constriction in accordance with one or more embodiments of the invention may include a method of (or apparatus for) treating bronchial constriction associated with anaphylactic shock, COPD or asthma, comprising applying at least one electrical impulse to one or more selected nerve fibers of a mammal in need of relief of bronchial constriction. The method may include: introducing one or more electrodes to the selected regions near or adjacent to the selected nerve fibers near or around the carotid sheath; and applying one or more electrical stimulation signals to the electrodes to produce the at least one electrical impulse, wherein the one or more electrical stimulation signals are of a frequency between about 15 Hz to 50 Hz.

The one or more electrical stimulation signals may be of an amplitude of between about 1-12 volts, depending on the size and shape of the electrodes and the distance between the electrodes and the selected nerve fibers. The one or more electrical stimulation signals may be one or more of a full or partial sinusoid, square wave, rectangular wave, and/or triangle wave. The one or more electrical stimulation signals may have a pulsed on-time of between about 50 to 500 microseconds, such as about 100, 200 or 400 microseconds. The polarity of the pulses may be maintained either positive or negative. Alternatively, the polarity of the pulses may be positive for some periods of the wave and negative for some other periods of the wave. By way of example, the polarity of the pulses may be altered about every second.

While the exact physiological causes of asthma, COPD and anaphylaxis have not been determined, aspects of the the present invention postulate that the direct mediation of the smooth muscles of the bronchia is the result of activity in one or more nerves near or in the carotid sheath. In the case of asthma, it appears that the airway tissue has both (i) a hypersensitivity to the allergen that causes the overproduction of the cytokines that stimulate the cholinergic receptors of the nerves and/or (ii) a baseline high parasympathetic tone or a high ramp up to a strong parasympathetic tone when confronted with any level of cholenergic cytokine. The combination can be lethal. Anaphylaxis appears to be mediated predominantly by the hypersensitivity to an allergen causing the massive overproduction of cholenergic receptor activating cytokines that overdrive the otherwise normally operating vagus nerve to signal massive constriction of the airways. Drugs such as epinephrine drive heart rate up while also relaxing the bronchial muscles, effecting temporary relief of symptoms from these conditions. Experience has shown that severing the vagus nerve (an extreme version of reducing the parasympathetic tone) has an effect similar to that of epinephrine on heart rate and bronchial diameter in that the heart begins to race (tachycardia) and the bronchial passageways dilate.

In accordance with various aspects of the present invention, the delivery, in a patient suffering from severe asthma, COPD or anaphylactic shock, of an electrical impulse sufficient to stimulate, block and/or modulate transmission of signals will result in relaxation of the bronchi smooth muscle, dilating airways and/or counteract the effect of histamine on the vagus nerve. Depending on the placement of the impulse, the stimulating, blocking and/or modulating signal can also raise the heart function.

Stimulating, blocking and/or modulating the signal in selected nerves in or around the carotid sheath to reduce parasympathetic tone provides an immediate emergency response, much like a defibrillator, in situations of severe asthma or COPD attacks or anaphylactic shock, providing immediate temporary dilation of the airways and optionally an increase of heart function until subsequent measures, such as administration of epinephrine, rescue breathing and intubation can be employed. Moreover, the teachings of various aspects of the present invention permit immediate airway dilation and/or heart function increase to enable subsequent life saving measures that otherwise would be ineffective or impossible due to severe constriction or other physiological effects. Treatment in accordance with one or more embodiments of the present invention provides bronchodilation and optionally increased heart function for a long enough period of time so that administered medication such as epinephrine has time to take effect before the patient suffocates.

In a preferred embodiment, a method of treating bronchial constriction comprises stimulating selected nerve fibers responsible for reducing the magnitude of constriction of smooth bronchial muscle to increase the activity of the selected nerve fibers. Certain signals of the parasympathetic nerve fibers cause a constriction of the smooth muscle surrounding the bronchial passages, while other signals of the parasympathetic nerve fibers carry the opposing signals that tend to open the bronchial passages. Specifically, it should be recognized that certain signals, such as cholinergic fibers mediate a response similar to that of histamine, while other signals (e.g., inhibitory nonadrenergic, noncholinergic or iNANC nerve fibers) generate an effect similar to epinephrine. Given the postulated balance between these signals, stimulating the iNANC nerve fibers and/or blocking or removing the cholinergic signals should create an imbalance emphasizing bronchodilation.

In one embodiment of the present invention, the selected nerve fibers are inhibitory nonadrenergic noncholinergic (iNANC) nerve fibers which are generally responsible for bronchodilation. Stimulation of these iNANC fibers increases their activity, thereby increasing bronchodilation and facilitating opening of the airways of the mammal. The stimulation may occur through direct stimulation of the efferent iNANC fibers that cause bronchodilation or indirectly through stimulation of the afferent sympathetic or parasympathetic nerves which carry signals to the brain and then back down through the iNANC nerve fibers to the bronchial passages.

In certain embodiments, the iNANC nerve fibers are associated with the vagus nerve and are thus directly responsible for bronchodilation. Alternatively, the iNANC fibers may be interneurons that are completely contained within the walls of the bronchial airways or extend from the esophagus to the trachea. These interneurons are responsible for modulating the cholinergic nerves in the bronchial passages. In this embodiment, the increased activity of the iNANC interneurons will cause inhibition or blocking of the cholinergic nerves responsible for bronchial constriction, thereby facilitating opening of the airways.

As discussed above, certain parasympathetic signals mediate a response similar to histamine, thereby causing a constriction of the smooth muscle surrounding the bronchial passages. Accordingly, the stimulating step is preferably carried out without substantially stimulating the parasympathetic nerve fibers, such as the cholinergic nerve fibers associated with the vagus nerve, that are responsible for increasing the magnitude of constriction of smooth muscle. In this manner, the activity of the iNANC nerve fibers are increased without increasing the activity of the adrenergic fibers which would otherwise induce further constriction of the smooth muscle. Alternatively, the method may comprise the step of actually inhibiting or blocking these cholinergic nerve fibers such that the nerves responsible for bronchodilation are stimulated while the nerves responsible for bronchial constriction are inhibited or completely blocked. This blocking signal may be separately applied to the inhibitory nerves; or it may be part of the same signal that is applied to the iNANC nerve fibers.

While it is believed that there are little to no direct sympathetic innervations of the bronchial smooth muscle in most individuals, recent evidence has suggested asthma patients do have such sympathetic innervations within the bronchial smooth muscle. In addition, the sympathetic nerves may have an indirect effect on the bronchial smooth muscle. Accordingly, alternative embodiments of the prevent invention contemplate a method of stimulating selected efferent sympathetic nerves responsible for mediating bronchial passages either directly or indirectly. The selected efferent sympathetic nerves may be nerves that directly innervate the smooth muscles, nerves that release systemic bronchodilators or nerves that directly modulate parasympathetic ganglia transmission (by stimulation or inhibition of preganglionic to postganglionic transmissions).

In one particular embodiment of the present invention, electrical impulses are delivered to one or more portions of the vagus nerve. The vagus nerve is composed of motor and sensory fibers. The vagus nerve leaves the cranium and is contained in the same sheath of dura matter with the accessory nerve. The vagus nerve passes down the neck within the carotid sheath to the root of the neck. The branches of distribution of the vagus nerve include, among others, the superior cardiac, the inferior cardiac, the anterior bronchial and the posterior bronchial branches. On the right side, the vagus nerve descends by the trachea to the back of the root of the lung, where it spreads out in the posterior pulmonary plexus. On the left side, the vagus nerve enters the thorax, crosses the left side of the arch of the aorta, and descends behind the root of the left lung, forming the posterior pulmonary plexus.

In mammals, two vagal components have evolved in the brainstem to regulate peripheral parasympathetic functions. The dorsal vagal complex (DVC), consisting of the dorsal motor nucleus (DMNX) and its connections, controls parasympathetic function below the level of the diaphragm, while the ventral vagal complex (VVC), comprised of nucleus ambiguus and nucleus retrofacial, controls functions above the diaphragm in organs such as the heart, thymus and lungs, as well as other glands and tissues of the neck and upper chest, and specialized muscles such as those of the esophageal complex.

The parasympathetic portion of the vagus innervates ganglionic neurons which are located in or adjacent to each target organ. The VVC appears only in mammals and is associated with positive as well as negative regulation of heart rate, bronchial constriction, bronchodilation, vocalization and contraction of the facial muscles in relation to emotional states. Generally speaking, this portion of the vagus nerve regulates parasympathetic tone. The VVC inhibition is released (turned off) in states of alertness. This in turn causes cardiac vagal tone to decrease and airways to open, to support responses to environmental challenges.

The parasympathetic tone is balanced in part by sympathetic innervations, which generally speaking supply signals tending to relax the bronchial muscles so overconstriction does not occur. Overall, airway smooth muscle tone is dependent on several factors, including parasympathetic input, inhibitory influence of circulating epinephrine, iNANC nerves and sympathetic innervations of the parasympathetic ganglia. Stimulation of certain nerve fibers of the vagus nerve (upregulation of tone), such as occurs in asthma or COPD attacks or anaphylactic shock, results in airway constriction and a decrease in heart rate. In general, the pathology of severe asthma, COPD and anaphylaxis appear to be mediated by inflammatory cytokines that overwhelm receptors on the nerve cells and cause the cells to massively upregulate the parasympathetic tone.

The methods described herein of applying an electrical impulse to a selected region of the vagus nerve may further be refined such that the at least one region may comprise at least one nerve fiber emanating from the patient's tenth cranial nerve (the vagus nerve), and in particular, at least one of the anterior bronchial branches thereof, or alternatively at least one of the posterior bronchial branches thereof. Preferably the impulse is provided to at least one of the anterior pulmonary or posterior pulmonary plexuses aligned along the exterior of the lung. As necessary, the impulse may be directed to nerves innervating only the bronchial tree and lung tissue itself. In addition, the impulse may be directed to a region of the vagus nerve to stimulate, block and/or modulate both the cardiac and bronchial branches. As recognized by those having skill in the art, this embodiment should be carefully evaluated prior to use in patients known to have preexisting cardiac issues.

Further testing on guinea pigs was made by applicant to determine the desired frequency range for reducing bronchoconstriction. These tests were all completed similarly as above by first establishing a consistent response to i.v. histamine, and then performing nerve stimulation at variations of frequency, voltage and pulse duration to identity parameters that attenuate responses to i.v. histamine. The tests were conducted on over 100 animals at the following frequency values: 1 Hz, 10 Hz, 15 Hz, 25 Hz, 50 Hz, 250 Hz, 500 Hz, 1000 Hz, 2000 Hz and 3000 Hz at pulse durations from 0.16 ms to 0.4 ms with most of the testing done at 0.2 ms. In each of the tests, applicant attempted to achieve a decrease in the histamine transient. Any decrease was noted, while a 50% reduction in histamine transient was considered a significant decrease.

The 25 Hz signal produced the best results by far with about 68% of the animals tested (over 50 animals tested at this frequency) achieving a reduction in histamine transient and about 17% of the animals achieving a significant (i.e., greater than 50%) reduction. In fact, 25 Hz was the only frequency in which any animal achieved a significant decrease in the histamine transient. About 30% of the animals produced no effect and only 2% (one animal) resulted in an increase in the histamine transient.

The 15 Hz signal was tested on 18 animals and showed some positive effects, although not as strong as the 25 Hz signal. As shown, 7 of the animals (39%) demonstrated a small decrease in histamine transient and none of the animals demonstrated an increase in histamine transient. Also, none of the animals achieved a significant (greater than 50%) reduction as was seen with the 25 Hz signal.

Frequency ranges below 15 Hz had little to no effect on the histamine transient, except that a 1 Hz signal had the opposite effect on one animal (histamine transient actually increased indicating a further constriction of the bronchial passages). Frequency ranges at or above 50 Hz appeared to either have no effect or they increased the histamine transient and thus increased the bronchoconstriction.

These tests demonstrate that applicant has made the surprising and unexpected discovery that a signal within a small frequency band will have a clinically significant impact on reducing the magnitude of bronchial constriction on animals subject to histamine. In particular, applicant has shown that a frequency range of about 15 Hz to about 50 Hz will have some positive effect on counteracting the impact of histamine, thereby producing bronchodilation. Frequencies outside of this range do not appear to have any impact and, in some case, make the bronchoconstriction worse. In particular, applicant has found that the frequency signal of 25 Hz appears to be desirable and thus the preferred frequency as this was the only frequency tested that resulted in a significant decrease in histamine transient in at least some of the animals and the only frequency tested that resulted in a positive response (i.e., decrease in histamine transient) in at least 66% of the treated animals.

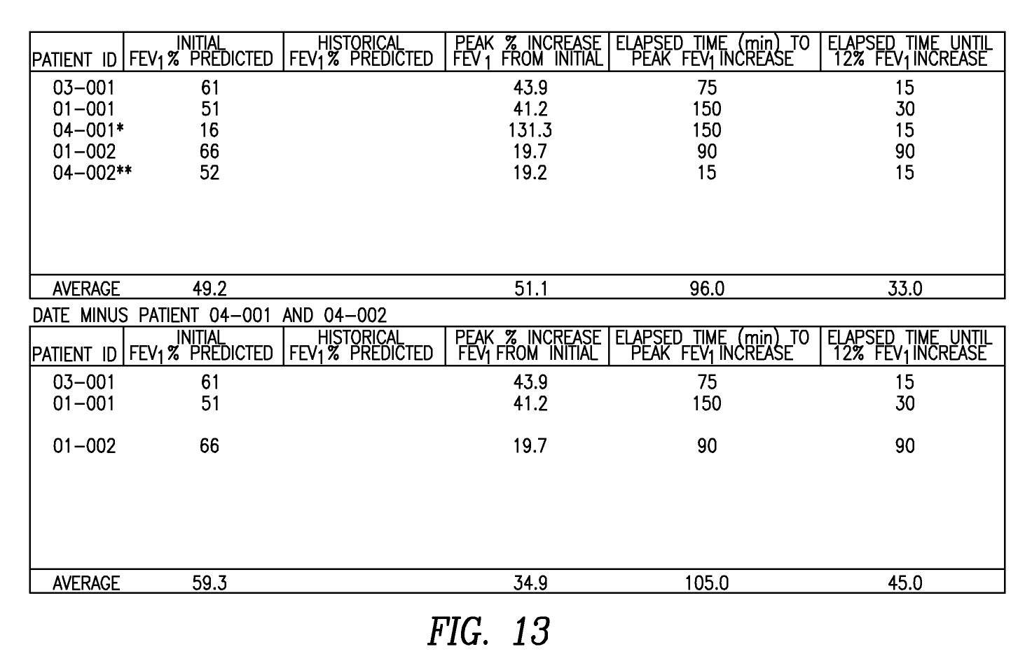

FIGS. 10-13 graphically illustrate exemplary experimental data obtained on five human patients in accordance with multiple embodiments of the present invention. In the first patient (see FIGS. 10 and 11), a 34 year-old, Hispanic male patient with a four year history of severe asthma was admitted to the emergency department with an acute asthma attack. He reported self treatment with albuterol without success. Upon admission, the patient was alert and calm but demonstrated bilateral wheezing, elevated blood pressure (BP) (163/92 mmHg) related to chronic hypertension, acute bronchitis, and mild throat hyperemia. All other vital signs were normal. The patient was administered albuterol (2.5 mg), prednisone (60 mg PO), and zithromax (500 mg PO) without improvement. The spirometry assessment of the lung function revealed a Forced Expiratory Volume in 1 second (FEV₁) of 2.68 1/min or 69% of predicted. Additional albuterol was administered without benefit and the patient was placed on supplemental oxygen (2 1/min).

A study entailing a new investigational medical device for stimulating the selected nerves near the carotid sheath was discussed with the patient and, after review, the patient completed the Informed Consent. Following a 90 minute observational period without notable improvement in symptoms, the patient underwent placement of a percutaneous, bipolar electrode to stimulate the selected nerves (see FIG. 16). Using anatomical landmarks and ultrasound guidance, the electrode was inserted to a position near the carotid sheath, and parallel to the vagus nerve.

The electrode insertion was uneventful and a sub-threshold test confirmed the device was functioning. Spirometry was repeated and FEV₁ remained unchanged at 2.68 1/min. Stimulation (25 Hz, 300 us pulse width signal) strength was gradually increased until the patient felt a mild muscle twitch at 7.5 volts then reduced to 7 volts. This setting achieved therapeutic levels without discomfort and the patient was able to repeat the FEV₁ test without difficulty. During stimulation, the FEV₁ improved immediately to 3.18 1/min and stabilized at 3.29 1/min (85% predicted) during 180 minutes of testing. The benefit remained during the first thirty minutes after terminating treatment, then decreased. By 60 minutes post stimulation, dyspnea returned and FEV₁ decreased to near pre-stimulation levels (73% predicted) (FIG. 2). The patient remained under observation overnight to monitor his hypertension and then discharged. At the 1-week follow-up visit, the exam showed complete healing of the insertion site, and the patient reported no after effects from the treatment.

This was, to the inventor's knowledge, the first use of nerve stimulation in a human asthma patient to treat bronchoconstriction. In the treatment report here, invasive surgery was not required. Instead a minimally invasive, percutaneous approach was used to position an electrode in close proximity to the selected nerves. This was a relatively simple and rapid procedure that was performed in the emergency department and completed in approximately 10 minutes without evidence of bleeding or scarring.

FIG. 12 graphically illustrates another patient treated according to one or more aspects of the present invention. Increasing doses of methacholine were given until a drop of 24% in FEV₁ was observed at 1 mg/ml. A second FEV₁ was taken prior to insertion of the electrode. The electrode was then inserted and another FEV₁ taken after electrode insertion and before stimulation. The stimulator was then turned on to 10 V for 4 minutes, the electrode removed and a post-stimulation FEV₁ taken showing a 16% increase. A final rescue albuterol treatment restored normal FEV₁.

FIG. 13 is a table summarizing the results of all five human patients. In all cases, FEV₁ values were measured prior to administration of the electrical impulse delivery to the patient according to one or more embodiments of the present invention. In addition, FEV₁ values were measures at every 15 minutes after the start of treatment. A 12% increase in FEV₁ is considered clinically significant. All five patients achieved a clinically significant increase in FEV₁ of 12% or greater in 90 minutes or less, which represents a clinically significant increase in an acute period of time. In addition, all five patients achieved at least a 19% increase in FEV₁ in 150 minutes or less.

As shown, the first patient initially presented with an FEV₁ of 61% of predicted. Upon application of the electrical impulse described above, the first patient achieved at least a 12% increase in FEV₁ in 15 minutes or less and achieved a peak increase in FEV₁ of 43.9% after 75 minutes. The second patient presented with an FEV₁ of 51% of predicted, achieved at least a 12% increase in FEV₁ in 30 minutes or less and achieved a peak increase in FEV₁ of 41.2% after 150 minutes. The third patient presented with an FEV₁ of 16% of predicted, achieved at least a 12% increase in FEV₁ in 15 minutes or less and achieved a peak increase in FEV₁ of about 131.3% in about 150 minutes. However, it should be noted that this patient's values were abnormal throughout the testing period. The patient was not under extreme duress as a value of 16% of predicted would indicate. Therefore, the exact numbers for this patient are suspect, although the patient's symptoms clearly improved and the FEV₁ increased in any event. The fourth patient presented with an FEV₁ of predicted of 66%, achieved at least a 12% increase in FEV₁ in 90 minutes or less and achieved a peak increase in FEV₁ of about 19.7% in 90 minutes or less. Similarly, the fifth patient presented with an FEV₁ of predicted of 52% and achieved a 19.2% peak increase in FEV₁ in 15 minutes or less. The electrode in the fifth patient was unintentionally removed around 30 minutes after treatment and, therefore, a true peak increase in FEV₁ was not determined.

In another embodiment of the present invention, a method for acutely treating post-operative ileus by applying one or more electrical impulses in or around the carotid sheath is described. Ileus occurs from hypomotility of the gastrointestinal tract in the absence of a mechanical bowel obstruction. This suggests that the muscle of the bowel wall is transiently impaired and fails to transport intestinal contents. This lack of coordinated propulsive action leads to the accumulation of both gas and fluids within the bowel. Although ileus has numerous causes, the postoperative state is the most common scenario for ileus development. Frequently, ileus occurs after intraperitoneal operations, but it may also occur after retroperitoneal and extra-abdominal surgery. The longest duration of ileus has been reported to occur after colonic surgery.

In accordance with this embodiment, methods and apparatus for treating the temporary arrest of intestinal peristalsis provide for: inducing at least one of an electric current, an electric field and an electromagnetic field in or around the carotid sheath to modulate, stimulate and/or block nerve signals thereof such that intestinal peristalsis function is at least partially improved. Specifically, prior to, during or after the operation that causes ileus, an electrode assembly 500 is introduced through the patient's neck and advanced to the target region in or around the carotid sheath (in the manner discussed above with reference to the treatment of bronchoconstriction). Once positioned, one or more drive signals are produced from a source of electrical energy to deliver one or more electrical impulses to the active and return electrode 502, 504 sufficient to modulate, stimulate and/or block nerve signals thereof such that intestinal peristalsis function is improved.

The drive signals inducing the current and/or fields preferably have a frequency, an amplitude, a duty cycle, a pulse width, a pulse shape, etc. selected to influence the therapeutic result, namely modulating some or all of the nerve transmissions in and around the carotid sheath. By way of example, the parameters of the drive signal may include a sine wave profile having a frequency of about 10 Hz or greater, such as between about 10-200 Hz, between about 15 Hz to 120 Hz, between about 25 Hz to about 50 Hz, between about 40-65 Hz, and more preferably about 50 Hz. The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts. The electric or electromagnetic field may be administered for a predetermined duration, such as between about 5 minutes and about 1 hour, or between about 5 minutes and about 24 hours. A more complete description of the mechanisms for increasing motility can be found in commonly assigned co-pending U.S. patent application Ser. No. 12/246,605, which has previously been incorporated herein by reference.

In yet another embodiment, the present invention can be used for treatment of hypotension utilizing an electrical signal that may be applied to selected nerves in the carotid sheath, such as the vagus nerve, to temporarily stimulate, block and/or modulate the signals in the selected nerves. Aspects of the present invention also encompass treatment of pathologies causing hypotension, both chronic and acute hypotension, such as in patients with thyroid pathologies and those suffering from septic shock. This treatment of hypotension may accompany treatment for other conditions, such as bronchial constriction, that also may occur in situations of shock.

In this embodiment, the present invention contemplates an electrical impulse delivery device that can be introduced through a percutaneous penetration in the patient's neck to a target region in or around the carotid sheath (as described above). The electrical impulses may be applied to at least one selected region of the carotid sheath to stimulate, block and/or modulate signals to the smooth muscle surrounding blood vessels, causing them to constrict and raise blood pressure.

Although the invention is not limited by any theory of operation, in one or more embodiments of the present invention, it is believed that the impulses may be applied in such a manner that the myocardium is relaxed to reduce the baseline level of tonic contraction, vasoconstriction occurs to increase blood pressure, and/or in cases of some shock, the smooth muscle lining the bronchial passages is relaxed to relieve the spasms that occur, such as during anaphylactic shock. The electric field generated around the active and return electrodes creates a field of effect that permeates the target nerve fibers and causes the stimulating, blocking and/or modulating of signals to the subject muscles. A more complete description of the mechanisms for elevating blood pressure can be found in commonly assigned co-pending U.S. patent application Ser. No. 11/592,095, which has previously been incorporated herein by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for acutely treating a condition or symptom of a patient comprising:
introducing an electrode through a percutaneous penetration in a patient to a target location adjacent to, or in close proximity with, the carotid sheath of the patient; and
applying an electrical impulse through the electrode to a selected nerve to modulate the selective nerve and acutely treat the condition or symptom of the patient.

2. The method of claim 1 wherein the introducing step is carried out by inserting a cannula through a skin surface in a neck of the patient and advancing the cannula to the target location proximal to the carotid sheath.

3. The method of claim 2 wherein the introducing step further comprises advancing the electrode through the cannula to a position parallel to the carotid sheath.

4. The method of claim 2 further comprising advancing an active and a return electrode through the cannula and applying an electrical voltage across the active and return electrodes.

5. The method of claim 1 further comprising stimulating one or more selected nerve fibers responsible for bronchial smooth muscle dilation to increase the activity of said nerve fibers.

6. The method of claim 1 wherein the electrical impulse is sufficient to acutely reduce a magnitude of bronchial constriction in a patient.

7. The method of claim 1 wherein the electrical impulse is of a frequency between about 15 Hz to 50 Hz.

8. The method of claim 1 wherein the electrical impulse is of an amplitude of between about 1 to 12 volts.

9. The method of claim 1 wherein the electrical impulse has a pulsed on-time of between about 50 to 500 microseconds.

10. The method of claim 1 wherein the selected nerve fibers are nonadrenergic noncholinergic nerve fibers.

11. The method of claim 1 further comprising increasing a blood pressure of the patient during the applying step.

12. The method of claim 1 further comprising at least partially improving peristalsis function during the applying step.

13. The method of claim 1 wherein the electrical impulse is sufficient to trigger an improvement in the patient's condition or symptom in less than 3 hours.

14. The method of claim 1 wherein the electrical impulse is sufficient to trigger an improvement in the patient's condition or symptom in less than 1 hour.

15. The method of claim 1 wherein the electrical impulse is sufficient to trigger an improvement in the patient's condition or symptom in less than 15 minutes.

16. A device for acutely treating a condition of a patient comprising:
a source of electrical energy;
an introducer configured for creating percutaneous access to a target region adjacent to, or in close proximity with, a carotid sheath of a patient; and
an electrode assembly coupled to the source of electrical energy and comprising at least one electrode sized for advancing to the target region, wherein the source of electrical energy is configured to apply an electrical impulse through the electrode to a selected nerve at the target region sufficient to modulate the nerve and treat the condition of the patient.

17. The device of claim 16 wherein the introducer comprises:
an access device for creating percutaneous access through a skin surface of a neck of the patient to the target region; and
a cannula having an inner lumen.

18. The device of claim 16 wherein the electrode assembly comprises an active electrode, a return electrode and electrical leads coupling the active and return electrodes to the source of electrical energy.

19. The device of claim 16 wherein the access device comprises a needle.

20. The device of claim 16 wherein the electrical impulse is sufficient to stimulate one or more selected nerve fibers responsible for smooth muscle dilation to increase the activity of said nerve fibers.

21. The device of claim 16 wherein the electrical impulse is sufficient to acutely reduce a magnitude of bronchial constriction in a patient.

22. The device of claim 16 wherein the electrical impulse is of a frequency between about 15 Hz to 50 Hz.

23. The device of claim 16 wherein the electrical impulse is of an amplitude of between about 1 to 12 volts.

24. The device of claim 16 wherein the electrical impulse has a pulsed on-time of between about 50 to 500 microseconds.
